(19) United States
(12) Patent Application Publication
(10) Pub. No.: US 2011/0001927 A1
(43) Pub. Date: Jan. 6, 2011

(54) FUNDUS SCANNING APPARATUS

(75) Inventor: Axel Kasper, Munich (DE)

Correspondence Address: ECKERT SEAMANS CHERIN & MELLOTT, LLC, U.S. STEEL TOWER, 600 GRANT STREET, PITTSBURGH, PA, 15219-2788, US

(73) Assignee: Linos Photonics GmbH & Co., KG, Goettingen (DE)

(21) Appl. No.: 12/865,477

(22) PCT Filed: Jan. 30, 2009
(86) PCT No.: PCT/EP09/51066

§ 371 Date: Aug. 23, 2010

(30) Foreign Application Priority Data
Feb. 1, 2008 (DE) 10 2008 000 225.9

(51) Int. Cl. A61B 3/14

(52) U.S. Cl. 351/206

(57) ABSTRACT

A combined fundus scanning apparatus for optical coherence tomography (OCT) and fundus imaging has an OCT unit (1) with a first light source creating a point-shaped scanning beam, an interferometer with a first sensor for sensing and evaluating the beam reflected by the retina (8), an imaging device (14), which focuses the scanning beam onto the retina (8) of an eye (9) to be examined, as well as a first beam deflection unit (4) to move the scanning beam in the X direction and a second beam deflection unit (11) to move the beam in the Y direction across the retina (8). Fundus imaging is effected by means of a second light source (5) that emits light of a spectral range different from the first light source, which light is also imaged onto the retina (8) via the imaging device (14), and which impinges on a second sensor (10) for sensing the light of the second light source (5) reflected by the retina (8). The second light source (5) is configured as a line light source, and the second sensor (10) is a line sensor. The light of the second light source (5) passes through one of beam deflection units (4, 11) to be moved by it across the retina (8).

The present invention relates to a combined fundus scanning apparatus for optical coherence tomography OCT and fundus imaging according to the preamble of claim 1.

Ophthalmoscopy is for diagnosing the fundus of the eye, wherein, in particular, the retina and the blood vessels supplying it are examined.

Existing optical systems for fundus imaging comprise, on the one hand, optical coherence tomographs, wherein the boundary surfaces and the tissue to be examined and therefore the tissue itself, such as the retina, can be scanned to a depth of a few millimeters by means of temporally short coherent light with the aid of an interferometer on the basis of the delay difference of two beams, wherein one of them is reflected on these boundary surfaces. It is thus possible to image the fine layered structure of the retina in specific areas.

There are also so-called fundus cameras and so-called scanning laser ophthalmoscopes or retinal scanners, which provide precise, high definition, surface images of large areas of the surface of the fundus of the eye. With the fundus cameras, the fundus of the eye is illuminated with the aid of radiation emitted by a light source and, with the aid of light reflected or emitted therefrom, imaging onto a surface sensor is carried out via an intermediate image. The high definition surface sensors to be used therefor are relatively expensive, however, to manufacture.

In a so-called scanning laser ophthalmoscope or retinal scanner the fundus of the eye is not illuminated over a large area but scanned with a focused light beam, and the reflected light is sensed by the sensor and allocated to the scanning sequence. A drawback of this method is, however, that the structure required therefor is relatively complex and expensive and that there is a time delay due to the point-to-point scanning, which leads to distorted results, due to, in particular, eye movements.

There are, however, also ophthalmic diagnosis devices that are a combination of a coherence tomograph and a scanning laser opthalmoscope and, as combined systems, offer a substantial advantage over two individual diagnosis devices. US 2006/0158655, for example, describes the combination of an optical coherence tomograph and a scanning laser ophthalmoscope. In addition to the problem of limited scanning speed, such systems have the drawback that the possibility of using a laser light source is greatly restricted or entails high costs, and therefore there is not a great range of light available for diagnosis.

Furthermore, there are combinations of optical coherence tomographs and traditional fundus cameras, which carry out imaging onto a high definition CCD sensor. These systems, as described, for example, in EP 1 808 119 A1, have an extremely complex structure and simply add up the drawbacks as well as the advantages of the fundus camera and optical coherence tomograph without utilizing synergistic effects.

It is thus the object of the present invention to provide a combination of an optical coherence tomograph and a fundus camera which utilizes synergistic effects to achieve cost effectiveness and to reduce the number of required components and which, at the same time, can supply a diagnostically useful high definition fundus image in real time.

This object is achieved according to the present invention by a combined fundus scanning apparatus for optical coherence tomography OCT and fundus imaging having the features of claim 1.

According to the present invention, the apparatus combines an OCT scanner for point-wise scanning of the retina in the XY direction, with a retinal scanner, which scans the retina on a line-per-line basis. A beam deflection unit of the XY scanner of the OCT is used to deflect the scanning line of the retinal scanner. All optical elements present downstream of this shared beam deflection unit in the beam direction serving for imaging the scanning beams onto the retina can also be shared by the two imaging systems, i.e. the OCT scanner and the retinal scanner. Due to the retinal scanner having a line light source and a line sensor, line-per-line scanning of the eye can thus be carried out by means of a beam deflection unit of the OCT scanner. A large portion of the imaging elements of the OCT scanner can thus be shared by the retinal scanner so that there are substantial synergistic effects. At the same time, this structure ensures that the scanning speed of the retinal scanner is within acceptable limits, since it does not use time consuming point-per-point scanning, but line based scanning, while high quality diagnostic fundus imaging can be produced. It is thus ensured that the two scanning systems in the apparatus according to the present invention have great synergistic effects so that substantial cost reduction is ensured while, at the same time, sufficiently high scanning speed of the fundus camera can be achieved, which allows a high definition fundus image to be produced in quasi real time.

Preferably, the deflection of the scanning line of the retinal scanner is carried out by means of the second beam deflection of the OCT scanner as seen in the beam direction of the OCT scanner. It moves both the one scanning direction of the OCT scanner as well as the scanning line produced by the line light source of the retinal scanner vertical to the line direction across the retina of the eye. This arrangement offers the possibility of a further advantageous embodiment wherein the first beam deflection unit in the beam direction of the OCT scanner is formed as a dichroic mirror so that the light of the line light source of the retinal scanner can be coupled into the beam path of the OCT scanner via the first beam deflection unit. Since the first beam deflection unit takes over the coupling-in function, the same beam path can be used for the retinal scan as for the OCT scan without an additional beam splitting element being necessary for coupling-in.

In an advantageous embodiment, the fast scan mirror of the OCT scanner is used for deflecting the scanning line of the retinal scanner. Due to the high repeat frequency of the fast scan it is possible to produce a real time image of the fundus even when the scan mode of the OCT scanner is not extremely fast. However, a sensor line that can be extremely rapidly read out is necessary for this purpose. Alternatively, the sensor line can be periodically read out, i.e. it is only ever read out after a certain number of scans, depending on how fast the sensor line is. This allows an extremely high image frequency to still be achieved whereby the information is, however, slightly blurred.

In a further advantageous embodiment, the slow scan mirror of the OCT scanner is thus used for deflecting the scan line of the retinal scanner. Although this causes the image frequency to be lower, it can still be sufficient with a fast OCT scanner.

In a further advantageous embodiment, the light source of the retinal scanner is implemented as an LED line. An LED line excellently allows a very narrow, relatively long and sufficiently homogeneous line light source to be realized, which often forms a sufficiently narrow line. A particular advantage of this LED line light source is that it can dispense with additional, expensive beam shaping optical elements, such as cylindrical lenses or the like. Due to the use, according to the present invention, of a line light source, relatively low power light can be used for working within the eye, making the LEDs an attractive light source option. They are a low-cost light source also suitable for use in an apparatus in doctor's practices. A very cheap light source can thus be realized that offers all options necessary for an excellently equipped flexible fundus scanning apparatus. Interference due to illumination back reflexes, in particular, is largely avoided.

In a particularly advantageous embodiment, a further advantage of the LED as a light source is utilized. By arranging different color LEDs in a line thus forming a light source a multicolor light source can be particularly easily realized, which enables the apparatus according to the present invention to be used for many, if not all, usual examination procedures in which retinal scanners are used. The suitable selection of LEDs or suitable filtering of white LEDs, for example, can thus enable both imaging of color images, red-free, infrared or autofluorescence images, and carrying out of methods, such as fluorescence angiography and indocyanine green angiography. It is, in particular, this combination of a line scanner for variable-color fundus imaging and illumination due to a line light source of multicolor LEDs from which almost any spectral range can be selected, which offers a particularly advantageous application of the apparatus. The images can be continuously recorded as video films in various spectral ranges. The fundus camera according to the present invention thus also enables carrying out angiography procedures that are particularly patient and user friendly. In a line scan, the patient is dazzled by substantially less light than in a surface scan. This is also why the pupil of the patient need not be dilated during the recording of a video film, allowing for a non-mydriatic procedure. Since a simple changeover between various illumination colors is possible with the LED illumination according to the present invention, it is thus also possible to carry out various angiographic procedures and OCT scans by means of a single multiple-use apparatus. In an advantageous embodiment, light of 765 nm for indocyanine green angiography, of 496 nm for fluorescence angiography and of 500 nm for autofluorescence angiography can thus be realized using white light or RGB LEDs as light sources via various filters able to be introduced into the beam path. The use of LEDs arranged in lines as a light source thus allows the retinal scanner to provide a relatively low-cost, durable system, into which an OCT scanner can be integrated with the multiple usage of optical components, and moreover, provides the flexibility and full functionability of a single retinal scanner deployable in all possible fields of use.

By arranging a preferably gap-shaped aperture in front of this light source, the line shape of this light source, already quite narrow, can be further restricted and optimized.

Preferably, the line sensor for the line scanning of the retinal scanner has a high definition configuration. Ideally, it comprises at least 1000 pixels in the line direction, so that sufficiently detailed fundus scanning is possible for diagnostic purposes. By moving the entire line across the retina by means of the beam deflection unit of the OCT scanner, it is possible to carry out rapid fundus scanning in spite of the large number of pixels, carried out in quasi real time. High definition sensors, in contrast to area sensors, can be realized relatively simply as line sensors and can thus be purchased relatively cheaply.

To make it possible to record a color image which, depending on the examination procedure, is to be recorded in a certain color range, the line sensor can also be realized as a color sensor. The desired color information is thus filtered out by the sensor itself without additional elements having to be present in the beam path. In a further advantageous embodiment, filters can be introduced into the beam path, which serve to filter out the color range desired for each examination procedure so that the sensor itself and also the light source need not be adapted to a particular spectral range as long as they comprise a sufficient number of color ranges. This is often a better approach, in particular if a lot of different color images are to be made possible.

In a further advantageous embodiment, the line sensor is implemented as a particularly cheap monochromatic sensor and the LEDs are sequentially switchable in various colors so that different color images for the respective examination procedures can be created by means of the illumination itself. In this embodiment a further advantage of the LEDs is used, namely their rapid switchability.

In a further advantageous embodiment, the line light source and the line sensor are confocally arranged. This enables excellent suppression of optical information not coming from the focal plane, which markedly improves the image quality of the individual images.

Advantageously, polarizers are arranged between the line light source and the object to be imaged, and between the latter and the sensor line, wherein the polarizers are orthogonal with respect to each other, so-called crossed polarizers. Preferably, they are polarizers with a high degree of polarization. To filter out interfering reflexes, the fact is utilized that the reflection or backscattering on the retina to be imaged has a depolarizing effect on the light to be sensed while this is not true for many other interfering reflexes (e.g. of lens surfaces of the optical system or of the cornea).

To increase the transmission efficiency for the useful light, it is advantageous to use a polarizing beam splitter to separate the illumination and imaging beam paths of the retinal scanner.

In a further advantageous embodiment, the interferometer unit of the OCT scanner is housed separately from the scan unit for the OCT and retinal scanner. This allows this unit to be arranged spatially separate from the examination device, making the examination device, in front of which ultimately the patient is seated, smaller and thus more mobile and easier to handle.

Further details and advantages of the invention can be derived from the dependent claims with reference to the description of an exemplary embodiment explained in detail with reference to the drawing.

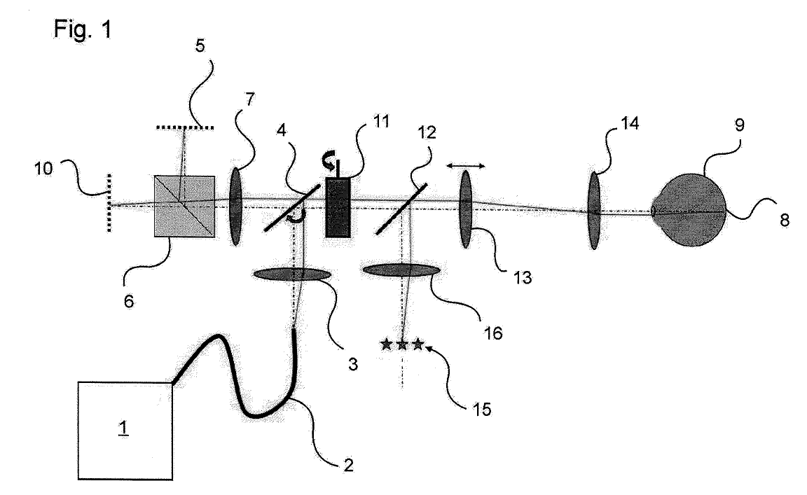

The single FIG. 1 schematically shows the structure of a combined fundus scanning apparatus for optical coherence tomography OCT and fundus imaging.

The combined retinal scanner with OCT and a diagnostic high definition fundus image shown in FIG. 1, has an OCT unit 1 for optical coherence tomography, comprising a first light source, an interferometer and a first sensor. The light from the first light source is guided from the interferometer output via a light guide 2 and a collimator 3 onto a dichroic scanner mirror 4 by which it is deflected. The light of an LED line 5 also impinges onto this dichroic scanner mirror 4, from the other side, and is imaged via a polarizing beam splitter 6 and a lens 7, and, after reflection on the retina 8 of eye 9, impinges on a line sensor 10, on which the fundus image is created. Downstream from the dichroic scanner mirror 4, as seen in the beam direction, the beams of the light source of OCT unit 1 and LED line 5 are co-extensive and impinge on a second scanner mirror 11, a dichroic accommodation beam splitter 12 for separating out the accommodation beam path, a dioptric lens 13 for adapting the camera to the individual, perhaps ametropic eyesight of the patient, and an objective lens 14, through which imaging is carried out onto retina 8. In the accommodation beam path, there is a fixing target 15 and a further imaging lens 16, which focuses the light onto fixing target 15, before, via the dichroic accommodation beam splitter 15, it is also coupled into the beam path and imaged onto retina 8.

The point-shaped light beam coming from OCT unit 1, which is usually a laser beam, or the beam of a superluminescent diode, is deflected in the X direction on a line-per-line basis by dichroic scanner mirror 4. The beam emitted by multicolor LED line 5 and guided onto dichroic scanner mirror 4 via a polarizing beam splitter 6 and imaging lens 7, is already shaped as a line beam and is thus not further deflected by dichroic scanner mirror 4, but passes through it and is thus coupled into the beam path of the beam coming from OCT unit 1. The two beams, line-shaped downstream from dichroic scanner mirror 4, are deflected in the Y direction on scanner mirror 11, so that a surface can be scanned by each of them. They pass through dichroic accommodation beam splitter 12 onto a dioptric lens 13, by which the entire apparatus is adapted to the ametropia, if any, of eye 9, and are imaged, via objective lens 14, onto retina 8, on which the scanning is carried out. By scanning retina 8 using the line of light created by LED line 5 and moved by means of scanner mirror 11, the radiation of which is reflected on retina 8, a fundus image of the entire retina 8 or a section thereof is sensed on sensor line 10. For this purpose the light, which is reflected on retina 8, is guided back via the same imaging beam path and via the polarizing beam splitter 6 onto a line sensor 10, which can be a CCD, a CMOS or a photodiode line. Line sensor 10 is configured as a color sensor and can separate the multicolor light emitted by LED line 5 into all available desired color ranges. Fundus images can thus be provided in various colors for different well-known applications.

To preserve the very rapidly switchable LEDs of LED line 5 and thus maximize their useful life, they are always switched off as long as no fundus image needs to be created. Dazzling of the patient by fundus illumination is thus also reduced to a minimum. Since it is desirable to create a real-time fundus image, a high repeat frequency of the fundus image must be ensured. Thus it makes sense to configure the shared scanner mirror 11 as a fast scan mirror for the optical coherence tomography. A fast scan mirror can help to scan the light of LED line 5 extremely rapidly across retina 8. To be able to sense all images thereby created, line sensor 10 must either be of a type that can be extremely rapidly read out, thus having an extremely high repeat frequency, or it must be driven in such a manner that reading out is carried out only after a certain number of scans of retina 8. The images created in the intermediate period are summed and averaged thus creating a certain blur of the image in a disadvantageous manner. To avoid this, LED line 5 could be switched in such a manner that the LEDs are only switched on as long as it takes to scan retina 8 once with the fast scan device.

Beam splitter 6 is configured as a polarizing beam splitter and can be configured so that it is not exactly orthogonal or is at a slight angle, so that all back reflexes created on its side surfaces are filtered out as far as possible so as not to negatively affect the quality of the fundus image.

As a result of the beam path of OCT unit 1, the beam path for the fundus image, and the accommodation beam path sharing as many optical elements as possible, a relatively cost-effective retinal scanner can be created, which is able, however, to produce fundus images in high resolution and in different colors for different diagnostic applications.

### LIST OF REFERENCE NUMERALS

1 OCT unit
2 light guide
3 collimator
4 dichroic scanner mirror
5 LED line
6 beam splitter
7 imaging lens
8 retina
9 eye
10 line sensor
11 scanner mirror
12 accommodation beam splitter
13 dioptric lens
14 objective lens
15 fixing target
16 imaging lens

What is claimed is:

1. A combined fundus scanning apparatus for optical coherence tomography (OCT) and fundus imaging, comprising:
an OCT unit including a first light source that creates a point-shaped scanning beam, an interferometer with a first sensor for imaging and evaluating the beam reflected by the retina, an imaging device that focuses the scanning beam onto the retina of an eye to be examined, a first beam deflection unit (4) for moving the scanning beam in the X direction and a second beam deflection unit for moving the scanning beam in the Y direction across the retina, and
a second light source that emits light of a spectral range different from that of the first light source, which is also imaged onto the retina via the imaging device, and a second sensor (10) for sensing the light of the second light source (5) reflected by the retina (8),
the improvement wherein, the second light source is configured as a line light source and the second sensor is a line sensor, and wherein the light of the second light source passes through one of the beam deflection units to be scanned across the retina by the latter.

2. The combined fundus scanning apparatus according to claim 1, wherein the beam deflection of the light of the second light source is carried out by the second beam deflection unit.

3. The combined fundus scanning apparatus according to claim 2, wherein the first beam deflection unit is of a dichroic type, so that the light of the second light source can thereby be coupled into the beam path of the first light source.

4. The combined fundus scanning apparatus according to claim 1, wherein the beam deflection of the light of the second light source is carried out by the beam deflection unit that carries out the fast scan of the first light source.

5. The combined fundus scanning apparatus according to claim 1, wherein the beam deflection of the light of the second light source is carried out by the beam deflection unit that carries out the slow scan of the first light source.

6. The combined fundus scanning apparatus according to claim 1, wherein the second light source is configured as an LED line.

7. The combined fundus scanning apparatus according to claim 6, wherein the second light source includes a gap-shaped aperture.

8. The combined fundus scanning apparatus according to claim 6, wherein the second light source includes multi-colored LEDs.

9. The combined fundus scanning apparatus according to claim 8, further comprising a selected one of a plurality of different color filters disposed in the beam path.

10. The combined fundus scanning apparatus according to claim 1, wherein the line sensor is of a high-definition type.

11. The combined fundus scanning apparatus according to claim 10, wherein the line sensor comprises at least 1000 pixels.

12. The combined fundus scanning apparatus according to claim 11, wherein the line sensor is a color sensor.

13. The combined fundus scanning apparatus according to claim 11, wherein the line sensor is a monochromatic sensor and color images are created by sequential illumination by the second light source.

14. The combined fundus scanning apparatus according to claim 1, wherein the second light source and the line sensor are confocally arranged.

15. The combined fundus scanning apparatus according to claim 1, wherein a polarizer is arranged in the illumination beam path and a polarizer is arranged in the imaging beam path of the second light source.

16. The combined fundus scanning apparatus according to claim 1, wherein a polarized beam splitter is arranged in the illumination and imaging beam paths of the second light source.

17. The combined fundus scanning apparatus according to claim 1, wherein the OCT unit is accommodated in a separate housing, which is connected with the fundus scanning apparatus by means of a light guide.
